(19) United States
(12) Patent Application Publication
(10) Pub. No.: US 2008/0269800 A1
(43) Pub. Date: Oct. 30, 2008

(54) ARTERIOTOMY CLOSURE SYSTEM WITH DUAL LUMENS SHEATH

(75) Inventors: Matthew Spurchise, Peabody, MA (US); Juan-Pablo Mas, Somerville, MA (US); Jeffrey J. Witts, North Reading, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/739,398

(22) Filed: Apr. 24, 2007

(51) Int. Cl. A61B 17/08

(52) U.S. Cl. 606/213

(57) ABSTRACT

An arteriotomy closure system includes a double barrel sheath having parallel dedicated lumens that merge at a distal juncture into a single common lumen. One of the dedicated lumens receives a dilator and the other a stapler. One of the dilator or stapler can be advanced from its dedicated lumen distally through the common lumen and beyond the distal end of the sheath while the other of the dilator or stapler is retracted into its dedicated lumen.

### FIELD OF THE INVENTION

The invention relates to systems for closing a percutaneous puncture in a blood vessel during a vascular procedure.

### BACKGROUND

Various cardiovascular procedures, such as angioplasty, stent placement and atherectomy, among others, are performed by inserting into and manipulating within the vasculator, wires and catheters adapted to perform those procedures. Access to the vasculature typically is through the femoral artery and is percutaneous, involving insertion of a needle and introducer sheath in the region of the groin to form a track and to puncture and create an arteriotomy in the femoral artery. A guidewire then is advanced through the needle and into the femoral artery. The needle then is removed. An introducer sheath is then advanced over the guidewire. The wire and sheath provide access into the femoral artery, through the arteriotomy, for catheters or other instrumentalities in order to perform the selected procedure.

After the procedure has been completed, the procedural devices are removed and the arteriotomy must be closed. A number of techniques are known to facilitate closure and healing of the arteriotomy. These include application of pressure at the puncture site for a relatively extended length of time or the use of biological adhesives or plugs adapted to seal the arteriotomy, among others. Also among the techniques for closing the arteriotomy is the use of a staple system such as described in U.S. Pat. Nos. 6,506,210, 6,767,356 and 7,074,232 to Kanner et al., of which the disclosures of the devices and methods are hereby incorporated by reference. The Kanner patents describe a system by which the original introducer sheath is removed, leaving the guidewire in place. Then, an assembly that includes a sheath and dilator is advanced along the indwelling guidewire to bring the distal end of the sheath into proximity to the arteriotomy. The sheath also carries an arrangement of wire-like stabilizers that, together with the dilator, pass through the arteriotomy into the artery. The system enables the portions of the stabilizer wires disposed within the artery to be formed into a temporarily enlarged shape that prevents removal of the wires through the arteriotomy. The stabilizers and distal end of the sheath are drawn together to grip the tissue about the arteriotomy and thereby secure and fix the position of the distal end of the sheath over and in alignment with the arteriotomy. The dilator and guidewire then are removed through the sheath, leaving the sheath in place adjacent the outer surface of the artery with the stabilizers within the artery, holding the sheath in place in readiness to provide direct access to the arteriotomy.

A catheter-like stapling device, with a staple carrier in its distal end, then is inserted into and advanced through the sheath to locate the staple in proximity to the arteriotomy. As described more fully in the Kanner patents, the sheath and stabilizer mechanisms orient the staple in registry with and at a fixed distance from the arteriotomy. When the stapler is actuated, the prongs of the staple expand and advance toward and into the arterial wall and surrounding tissue on opposite sides of the arteriotomy. The stapling mechanism then draws the prongs of the staple together to draw the edges of the arteriotomy together into approximation and then releases the staple. The stabilizers are caused to return to a linear shape, enabling their withdrawal. With the staple deployed and having closed the arteriotomy, the stapling mechanism and sheath may be removed, leaving the staple in place.

In using the foregoing system, after the sheath has been positioned and stabilized, the dilator and indwelling guidewire must be withdrawn completely from the sheath in order to permit the stapler to be inserted into the sheath and then advanced to position the staple adjacent the arteriotomy. It would be desirable to reduce the number of steps and manipulations in this procedure and to simplify the procedure. It is among the general objects of the invention to do so.

### SUMMARY OF THE INVENTION

The invention includes the use of a sheath having a proximal elongate segment having a pair of separate, parallel, dedicated lumens that merge, at a juncture, into a single, common lumen that extends through a distal segment of the sheath to a distal opening. One of the dedicated lumens is adapted to receive the dilator and the other dedicated lumen receives the stapler. Either the dilator or stapler can be advanced or withdrawn through its dedicated proximal lumen and the single common lumen in the distal segment of the sheath while the other, stapler or dilator is withdrawn into its dedicated lumen.

When the device is used to close the arteriotomy, the procedural devices (e.g., catheter, introducer) will have been removed, leaving the indwelling guidewire. The device is presented with the dilator extending through and beyond the distal end of the common lumen and is backloaded over the indwelling guidewire. During this stage, the stapler is preloaded in its dedicated lumen, withdrawn proximally of the junction where the two lumens merge into the common lumen. The assembly is advanced until the distal end of the dilator has been positioned properly within the arterial lumen, the positioning being confirmed by the presence of blood at the proximal end of a blood marking lumen of the dilator. The position of the system with respect to the arteriotomy then is stabilized by wire-like stabilizers that are attached to and project distally from the distal end of the sheath, through the arteriotomy and into the lumen of the artery. With the dilator having been advanced sufficiently within the artery, as indicated by the blood marking indicator, the stabilizers are operated to assume a non-linear shape within the artery adapted to engage the inner surface of the artery lumen to stabilize the position of the sheath and prevent its dislodgement. With the sheath stabilized, the dilator can be withdrawn so that its distal end is located within its dedicated lumen. The stapler then can be advanced distally from its dedicated lumen into the common lumen of the sheath to place the staple adjacent the arteriotomy in readiness to cause the staple to engage and close the arteriotomy. Upon closure of the arteriotomy, the entire apparatus may be removed.

### DESCRIPTION OF THE DRAWINGS

In the accompanying drawings:

FIG. 1 is a diagrammatic fragmented illustration, partly broken away and in section, of a sheath embodying principles of the invention;

FIG. 1A is a partly sectional illustration as seen along the line 1A-1A of FIG. 1 showing the stabilizers in a longitudinally extended, linear configuration;

FIG. 1B is a sectional illustration as seen along the line 1B-1B of FIG. 1;

FIG. 1C is a sectional illustration as seen along the line 1C-1C of FIG. 1 and illustrating, somewhat diagrammatically, the position of the dilator and the stapler in their respective dedicated lumens;

FIG. 2A is an illustration of the system similar to FIG. 1 with enlarged detail and illustrating the system in readiness for use with the dilator extended beyond the end of the sheath and the staple retracted into its dedicated lumen;

FIG. 2B is an illustration of the system similar to FIG. 2A showing both the dilator and the stapler retracted into their respective dedicated lumens and with the stabilizing wires in a deployed configuration;

FIG. 2C is an illustration of the system similar to FIG. 2A showing the dilator retracted in its dedicated lumen and the stapler extended from its dedicated lumen through the common lumen and into a position in readiness to deploy the staple to close the arteriotomy;

FIGS. 3-5 are fragmented illustrations of a two prong staple and stapler as may be employed in practicing the invention with the staples being shown respectively in their delivery, open and closed positions;

FIG. 6 is a diagrammatic illustration of the distal end of the sheath disposed in proximity to the arteriotomy and with the stabilizers deployed within the artery, with the dilator having been retracted and in readiness to receive the stapler;

FIG. 7 is a diagrammatic plan view of the arteriotomy illustrating the manner in which the stabilizer wires engage the arteriotomy;

FIGS. 8A-8C illustrate, in enlarged detail, one embodiment of the stabilizers;

FIG. 9 is an illustration similar to FIG. 1 of another embodiment of the invention; and

FIG. 10 is a sectional illustration as seen along the line 10-10 of FIG. 9.

### DESCRIPTION OF ILLUSTRATIVE EMBODIMENTS

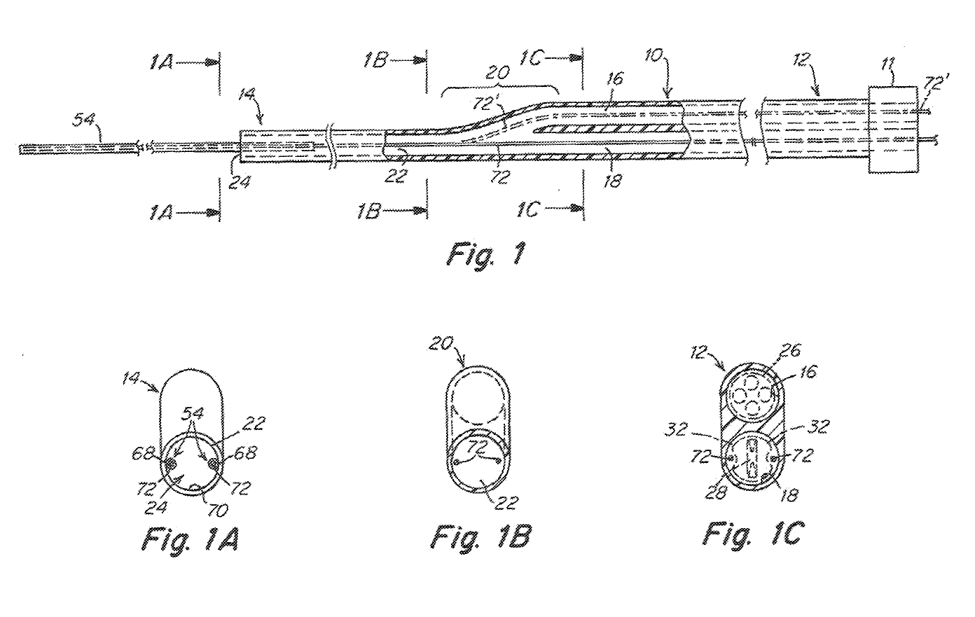

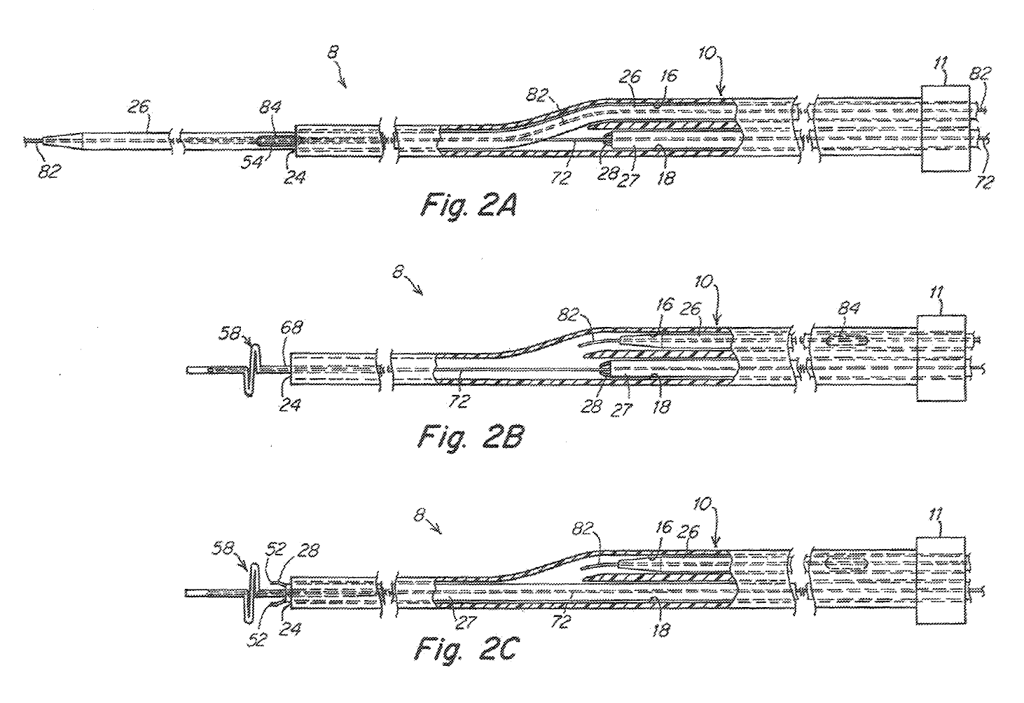

FIG. 1 illustrates, diagrammatically, one embodiment of a sheath 10 that form a part of the system and may be used in the practice of the invention. In this embodiment, the sheath may be formed from a suitable biocompatible polymer to include an elongate proximal segment 12 and a distal segment 14. The proximal portion 12 is formed to include two parallel lumens 16, 18 that merge at a juncture region 20 into a single distal lumen 22 that terminates in a distal outlet 24. One of the lumens 16 is dimensioned and dedicated to receive a dilator 26 (FIGS. 2A-2C), the other lumen 18 being configured and dedicated to receive a single-shot stapling device 27 adapted to carry a staple 28 and deploy the staple at the arteriotomy to close the arteriotomy. In the illustrative embodiment, the stapler and staple may be as described in pending U.S. patent application Ser. No. 11/626,616 filed Jan. 24, 2007, the disclosure of which is incorporated by reference herein, in its entirety. It should be understood, however, that other staplers adapted for closing an arteriotomy also may be used.

The proximal end of the sheath may include appropriate mechanisms, indicated generally at 11, by which the various instrumentalities of the system 8 may be controlled. For example, mechanism 11 may include a device or devices as described in further detail in U.S. Pat. No. 6,767,356 to actuate the system and control the movement and sequence of operation of its various components.

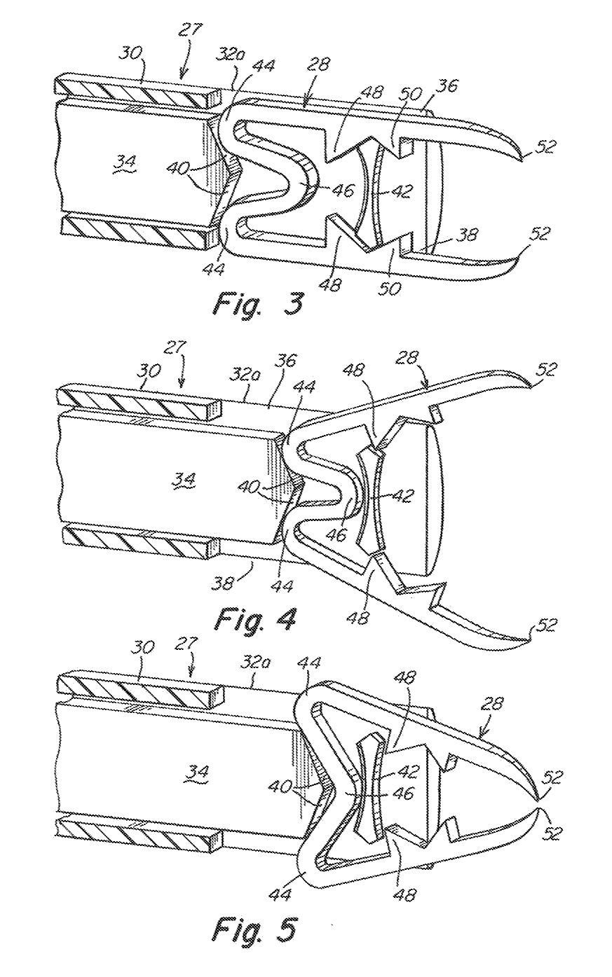

One such stapler 27 and staple 28, described in further detail in application Ser. No. 11/626,616, is illustrated in FIGS. 3-5 in which the staple 28 is a two prong staple that lies generally in a flat plane and in which the stapler 27 also has a somewhat flattened profile. The stapler 27 includes an elongate hollow shaft 30. A pair of flat-sided laterally placed actuation tips 32 (only one tip 32a is shown in FIGS. 3-5) is mounted at the distal end of the shaft 30. The flat sides of the actuation tips 32 face each other and define a passageway between them to define a chamber that receives the staple 28 and provides a passageway through which a flat distal portion of an elongate driver 34 may be advanced. The staple chamber is open at its top and bottom regions 36, 38. The distal end of the driver 34 has distally facing driving faces 40. An anvil 42 for forming or deflecting the staple is fixed within the staple chamber between the actuation tips.

The staple 28 includes a pair of legs having proximally located expansion bends 44 connected by a closure bend 46. Each leg of the staple has an expansion ramp 48 and a sharp tip 52 and may have a staple tissue stop 50.

The stapler is operated after it has been advanced through the lumens 18, 22 of the sheath, into a position adjacent the arteriotomy. The driver 34 then is advanced so that the drive faces 40 push the staple distally in the chamber to cause the expansion ramps 48 to ride along and be forced apart by the anvil 42. The staple legs separate and extend laterally through the open top and bottom 36, 38 of the chamber. Simultaneously, or closely coordinated with the spreading of the staple legs, the staple is advanced further distally to cause the tips 52 to pierce the tissue and vessel on each side of the arteriotomy. Continued advancement of the driver causes the staple closure bend 46 to abut the anvil 42 while the expansion ramps 48 clear the anvil 42. Driver faces 40 force closure bend 46 against the anvil thereby deforming the closure bend 46 in a more open angle that, in turn, causes the legs of the staple to pivot in opposite directions about the closure bend 46 to draw the legs together, closing the arteriotomy.

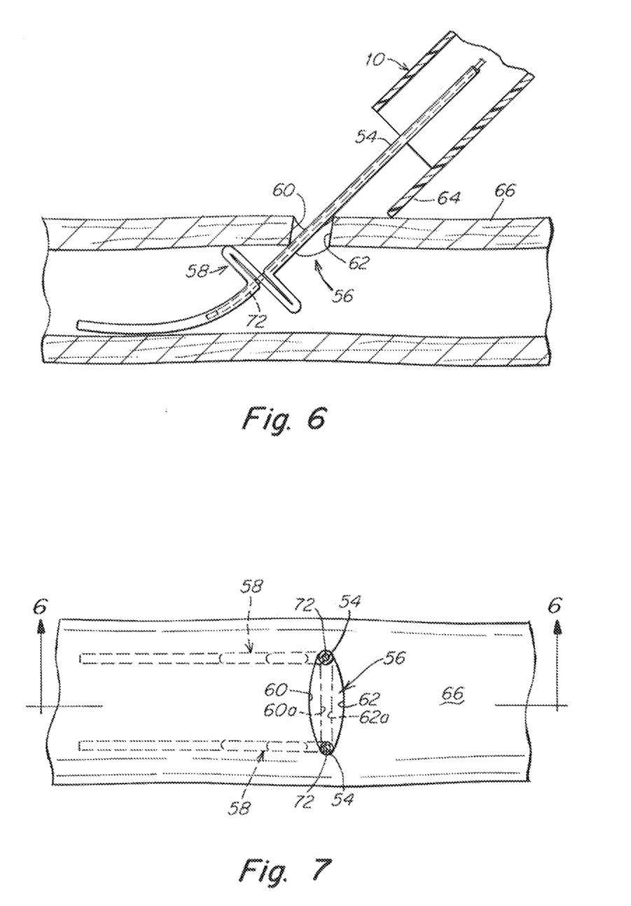

As shown diagrammatically in FIGS. 6 and 7, sheath 10 has wire-like stabilizers 54 that extend through and engage the ends of the arteriotomy 56 to position and align the distal end of the sheath 10 with the arteriotomy 56 and to stabilize it in that position for subsequent advancement and operation of the stapler 27. The stabilizers 54 may be mounted to the sheath 10 and may be operated to change configuration temporarily from a linear or low profile configuration to a non-linear, greater profile configuration, such as the zigzag arrangement 58 shown in FIG. 6. The stabilizers are mounted with respect to the sheath 10 so that they engage the lateral ends of the arteriotomy 56 and may be spaced to stretch the arteriotomy 56 laterally to tension the edges 60, 62 to draw them toward each other as suggested in phantom at 60a, 62a in FIG. 7. The non-linear configuration 58 of the stabilizers 54 serves to retain the stabilizers in place and prevents them from being withdrawn through the arteriotomy. Additionally, the sheath 10 may be provided with a gauge 64 that projects a fixed distance distally beyond the distal end of the sheath to engage the outer surface of the vessel 66 or surrounding tissue when the distal end of the sheath has reached that distance from the outer surface of the vessel 66. Thus, the foregoing arrangement serves to position the sheath 10 in proper alignment with the arteriotomy and the stabilizers 54 serve to approximate the edges 60, 62 of the arteriotomy in readiness for stapling.

In the illustrative embodiment of the invention, the stabilizers are coupled to the sheath 10 and extend distally beyond the distal outlet 24 of the sheath. As shown in FIG. 1A, the stabilizers may be in the form of a pair of flexible tubular members 68 attached to the sheath and extending from opposite sides of the inner luminal surface 70 of the common lumen 22. The stabilizers also include a control wire 72 that extends through the stabilizer tubes, the control wire being connected to the tubes 68 a few centimeters from their tips. The portion of the stabilizer tubes disposed outside of the sheath is formed with relatively weakened portions so that tensioning control wires 72 will compress the stabilizer tubes longitudinally to cause them to fold or otherwise assume an enlarged profile such as that indicated at 58 in FIG. 6. Although the control wires 72 are shown in FIGS. 1C and 2A-2C as being contained in the second (stapler) lumen 18, they may be passed, alternately, through the first dilator lumen 16 as suggested in phantom at 72′ in FIG. 1. In the alternate configuration the dilator and first lumen should be dimensioned and configured to enable freedom of movement of the wires 72′.

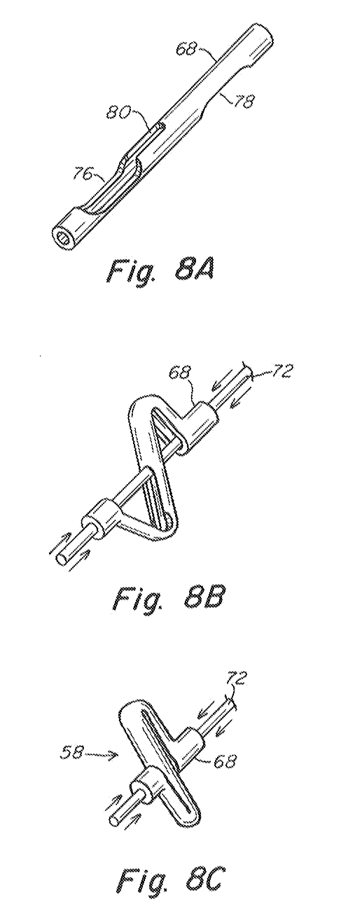

FIGS. 8A-8C illustrate, sequentially, one manner in which the stabilizers may be configured. The stabilizer tubes 68 may be formed with generally symmetrical notches 76 and 78 on either side, with slots 80 emanating from the notches and overlapping approximately midway between the notches. The slots 80 overlap forming a through hole approximately equal to the inside diameter of the tube. The tube in the area of that slot has a U-shaped cross-section. Compression causes the tubular member to fold at the notched sections 76 and 78, fulcruming on the wire at the location where the slots overlap.

FIG. 2A illustrates, diagrammatically, the manner in which the arteriotomy closure system, including sheath 10 preloaded with a dilator 26 and stapler 27, is backloaded onto and advanced over an indwelling guidewire 82 after the procedural devices have been removed. The stapler 27 is retracted in its dedicated lumen 18. The system is provided in readiness for use with the dilator 26 already extended distally beyond the end of the sheath 10. Side pockets 84 are formed in the dilator 26 and are adapted to receive the distal ends of the stabilizers so that the stabilizers 54 will not project laterally as the dilator is advanced through the tissue. Once the dilator and the distal ends of the stabilizers have reached the lumen of the vessel, the dilator is advanced slightly distally relative to the sheath, releasing the distal ends of the stabilizers 54 from the pockets 84 in which they had been concealed. The stabilizers then may be actuated by tensioning the control wires 72, to cause them to assume an enlarged, deployed configuration 58 (FIGS. 6 and 8C). With the stabilizers deployed, the distal end of the sheath 10 is aligned with the arteriotomy 56 (FIG. 6). The position of the dilator in the artery lumen is confirmed by the presence of blood passing through a blood marking lumen and flowing from a marker port (not shown) on the proximal end of the dilator exiting from sheath 10. The distal end of the sheath then can be drawn closer to the arteriotomy 56 so that the stabilizers and distal end of the sheath may grip the artery wall about the region of the arteriotomy (FIG. 6). With the distal end of the sheath positioned and secured with respect to the arteriotomy, the dilator 26 and guide wire 82 are retracted proximally out of the distal lumen 22 into dedicated lumen 16. The stapler 27 then can be advanced distally from its dedicated lumen 18 into the single distal lumen 22 and into position with respect to the arteriotomy so that the staple can engage tissue on opposite sides of the arteriotomy with the staple then being crimped to close the arteriotomy. The staple is released from the stapler and the device then is operated to straighten the stabilizers to a low profile or linear configuration (e.g., FIG. 1) so that they can be withdrawn from the arteriotomy. With the arteriotomy thus closed, the sheath 10, carrying the dilator and the stapler, is removed from the patient.

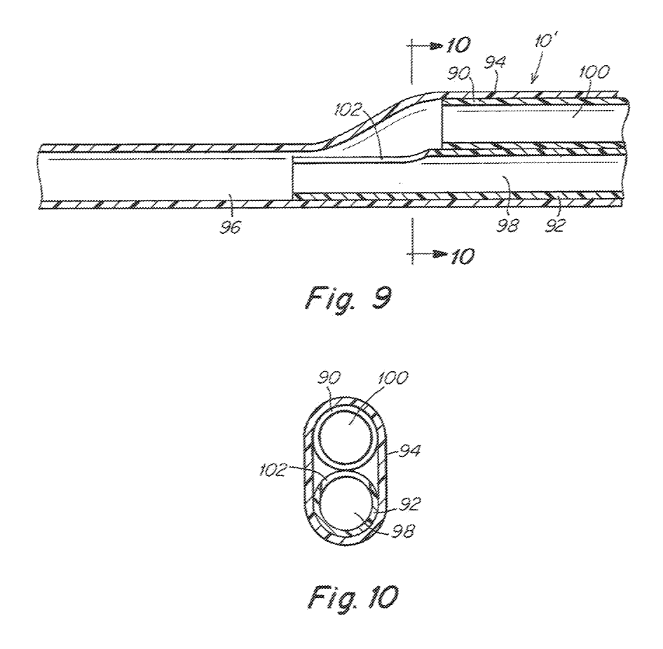

FIGS. 9 and 10 illustrate another embodiment of the sheath 10′ as may be adapted to the present invention. In this embodiment, the dedicated lumens may be formed from a pair of tubes 90, 92 to form the dedicated lumens. The tubes may be joined by a surrounding outer sleeve 94 that extends distally beyond the ends of the tubes to define the common lumen 96. The tubes 90, 92 may be arranged as shown in which the stapler lumen 98 extends distally beyond the dilator lumen 100. A notch 102 may be formed in the end of the stapler tube 92 to permit free passage of the dilator from its dedicated lumen 100 into the common lumen 96.

In both embodiments, it may be noted that the dedicated stapler lumen is substantially aligned with the common lumen in the distal segment in order to avoid requiring bending in the stapling device. The dilator, which is formed from a more flexible plastic material may deform sufficiently through the juncture of the proximal and distal segments.

It also should be understood that the foregoing description of the invention is intended merely to be illustrative and that other embodiments, modifications and equivalents may be apparent to those skilled in the art while remaining within the scope of the invention.

1. An arteriotomy closure system comprising:
an elongate sheath having proximal and distal segments, the sheath having parallel first and second dedicated lumens in the proximal segment, the dedicated lumens being joined at their distal ends at a juncture;
a common single lumen extending through the distal segment from the juncture to a distal outlet;
a dilator contained within the first dedicated lumen and the common lumen, the distal tip of the dilator extending through the outlet beyond the end of the sheath, the dilator having a guidewire lumen and a blood marking lumen;
the dilator being movable distally and proximally relative to the sheath to enable the distal end of the dilator to be withdrawn from the common lumen to a position proximal of the juncture;
a tissue stapler disposed within the second dedicated lumen with the distal end of the stapler being disposed proximally of the juncture; and
whereby after withdrawal of the dilator from the common lumen, the stapler then may be advanced into the common lumen to a position to staple the arteriotomy.

2. system as defined claim 1 wherein the second dedicated lumen is substantially axially aligned with the common lumen.

3. A system as defined in claim 1 wherein the sheath is formed substantially in its entirety from an integral polymeric material.

4. A system as defined in claim 1 wherein the sheath comprises a first tube and a second tube, the first tube and second tube extending parallel to each other, the first tube defining the first dedicated lumen and the second tube defining the second dedicated lumen;
a polymeric sleeve disposed about both tubes to retain the first and second tubes together, the sleeve having a distal segment that extends distally beyond the ends of the tubes and defining the common signal lumen.

5. A system as defined in claim 1 further comprising:
a pair of stabilizers fixed to the sheath and extending beyond the distal end of the sheath, the stabilizers being adapted to project into an artery through the arteriotomy, the stabilizers being transformable between a low profile and an enlarged profile whereby in their enlarged configuration they preclude withdrawal of the stabilizers from the artery.

6. A system as defined in claim 5 wherein the stabilizers are fixed to and protrude from the interior of the common lumen distally beyond the end of the common lumen.

7. A system as defined in claim 5 wherein each stabilizer comprises a flexible tube having a weakened portion adapted to assume an enlarged shape when compressed axially; and
a wire extending through the tube to enable compression of the wire, the wire extending through the common lumen and through one of the first or the second dedicated lumens, the profile of the stapler within its dedicated lumen providing a passageway for each of the wires.

8. A system as defined in claim 4 wherein the second tube is formed from a more rigid material than that of the first tube, the second tube having a distal end extending beyond the distal end of the first tube and having a notch formed in the distally extending portion of the second tube, the notch being adapted to provide a pathway from the first dedicated lumen to the common lumen.

9. A method for closing an arteriotomy in a vessel of a patient, the method comprising:
providing an elongated sheath having proximal and distal segments, and parallel first and second dedicated lumens in the proximal segment, the dedicated lumens being joined at their distal ends at a juncture, and a common single lumen extending through the distal segment from the juncture to a distal outlet;
providing a dilator having a blood marking lumen and a guidewire lumen, the dilator being disposed within the first dedicated lumen and the common lumen with a distal end of the dilator extending distally beyond the distal end of the sheath;
providing a stapler and a tissue staple disposed within the second dedicated lumen, a distal end of the stapler being disposed proximally of the juncture;
advancing the dilator together with the sheath over an indwelling guidewire that extends from an interior lumen of the vessel to the exterior of the patient until the blood marking lumen provides an indication that the dilator is positioned properly within the artery;
stabilizing the position of a distal end of the sheath with respect to the arteriotomy;
withdrawing the dilator and guidewire to position the distal ends of the guidewire and the dilator proximally of the juncture;
thereafter advancing the stapler distally into the common lumen to position its distal end adjacent the arteriotomy; and
actuating the stapler to staple the arteriotomy closed.
